(19) United States
(12) Patent Application Publication
(10) Pub. No.: US 2026/0287695 A1
(43) Pub. Date: Sep. 24, 2026

(54) METHOD AND SYSTEM FOR PERFORMING SCANS BY MULTIPLE IMAGING SYSTEMS

(71) Applicant: KONINKLIJKE PHILIPS N.V., EINDHOVEN (NL)

(72) Inventors: Shyam Bharat, Arlington, MA (US); Gary Ng, Redmond, WA (US); Claudia Errico, Medford, MA (US)

(21) Appl. No.: 19/472,374

(22) PCT Filed: Mar. 29, 2024
(86) PCT No.: PCT/EP2024/058754

§ 371 Date: Oct. 3, 2025

Related U.S. Application Data
(60) Provisional application No. 63/457,166, filed on Apr. 5, 2023.

(51) Int. Cl. G01R 33/48; G01N 23/046; G01R 33/30; G01S 15/86

(52) U.S. Cl. CPC G01R 33/4812; G01R 33/307; G01R 33/4814; G01N 23/046; G01R 33/481; G01S 15/86

(57) ABSTRACT

A system and method are provided for associating a first workflow of a first scan of a subject (160) using a first imaging system (140) and a second workflow of a second scan using a second imaging system (150). The method includes acquiring first scan images using the first imaging system (S211); acquiring first camera images using a first camera (145) (S212); generating a correspondence between the first scan images and surface locations on the subject (S213); extracting content from the first scan related to a region of interest from the first scan images (S214); generating a results report of the first scan using the extracted content (S215); creating a customized scan protocol for the second scan using the report (S221); acquiring second scan images of the subject during the second scan using the second imaging system according to the customized scan protocol (S222); acquiring second camera images using a second camera (155) (S223); and monitoring progress of the customized scan protocol using the second camera images (S224).

### BACKGROUND

Diagnostic imaging is pervasive in modern healthcare, including computed tomography (CT) scans, positron emission tomography (PET) scans, magnetic resonance imaging (MRI), and ultrasound (US) imaging, for example. Diagnostic workflows of the medical imaging often involve use of multiple imaging systems having different modalities, such as a CT or MRI as a first imaging system followed by ultrasound as a second imaging system to confirm or rule out findings from the first imaging system.

In current clinical practice, once a CT scan is performed as the first imaging system, for example, a radiologist evaluates the CT images and generates a CT scan report. The scan report may be sent to a referring physician, who decides whether a follow-up scan using a different system, e.g., ultrasound, is warranted. The referring physician sends out an exam order for the ultrasound scan to a sonography lab as the second imaging system, where the CT scan report may or may not be included with the exam order. A sonographer or other clinician performs the ultrasound scan in accordance with the description created by the referring physician in the exam order. However, the information transfer, including the CT scan report and/or CT images, is at a high level and subject to translation by the sonographer. Also, the second imaging system is performed using a standard protocol, which will not necessarily account for unique aspects of the CT scan as applied to a particular subject. Therefore, actions in the workflow of the second imaging system are not directly optimized based on findings in the first imaging system from an efficiency perspective.

### SUMMARY

According to a representative embodiment, a method is provided for performing a first scan of a subject using a first imaging system and a second scan of the subject using a second imaging system. The method includes acquiring first scan images of the subject during the first scan using the first imaging system; acquiring first camera images of a surface of the subject during the first scan using at least one first camera associated with the first imaging system; generating a correspondence between the first scan images acquired by the first imaging system and surface locations on the surface of the subject from which the first scan images are acquired, respectively; extracting content from the first scan related to a region of interest in the subject identified in the first scan images; and generating a report of results of the first scan based on the extracted content, where generating the report includes associating the first camera images with the first scan images, respectively, estimating a surface location on the surface of the subject corresponding to the region of interest using the first scan images and the first camera images, and indicating in the report the region of interest in the subject relative to the estimated surface location. The method further includes automatically creating a customized scan protocol for the second scan based on the report, where the customized scan protocol provides a plurality of imaging steps and a plurality of device settings of the second imaging system corresponding to the plurality of imaging steps, respectively; acquiring second scan images of the subject during the second scan using the second imaging system according to the plurality of imaging steps and the corresponding plurality of device settings of the customized scan protocol; acquiring second camera images of the surface of the subject during the second scan using at least one second camera associated with the second imaging system; and monitoring progress of the customized scan protocol during the second scan to identify performance of the plurality of imaging steps using the second camera images from the at least one second camera.

According to another representative embodiment, a system includes a first imaging system configured to acquire first scan images of a subject during a first scan; a second imaging system configured to acquire second scan images of the subject during a second scan subsequent to the first scan; at least one first camera associated with the first imaging system and configured to acquire first camera images of a surface of the subject during the first scan; at least one second camera associated with the second imaging system and configured to acquire second camera images of the surface of the subject during the second scan; at least one computer processor; and at least one memory storing instructions for associating workflows of the first scan the second scan. When executed, the instructions cause the at least one computer processor to generate a correspondence between the first scan images acquired by the first imaging system and surface locations on the surface of the subject from which the first scan images are acquired during the first scan, respectively; extract content from the first scan related to a region of interest in the subject identified in the first scan images; generate a report of results of the first scan based on the extracted content, where the report is generated by associating the first camera images with the first scan images, respectively, estimating a surface location on the surface of the subject corresponding to the region of interest using the first scan images and the first camera images associated with the first scan images, and indicating in the report the region of interest in the subject relative to the estimated surface location; create a customized scan protocol for the second scan based on the report, where the customized scan protocol provides a plurality of imaging steps and a plurality of device settings of the second imaging system corresponding to the plurality of imaging steps, respectively, where the second scan images of the subject are acquired during the second scan by the second imaging system according to the plurality of imaging steps and the corresponding plurality of device settings of the customized scan protocol; and monitor progress of the customized scan protocol during the second scan to identify performance of the plurality of imaging steps using the second camera images from the at least one second camera.

According to another representative embodiment, a non-transitory computer readable medium is provided storing instructions for performing a first scan of a subject using a first imaging system and a second scan of the subject using a second imaging system that. When executed by at least one processor, the instructions cause the at least one processor to receive first scan images of the subject acquired during the first scan using the first imaging system receive first camera images of a surface of the subject acquired during the first scan using at least one first camera associated with the first imaging system; generate a correspondence between the first scan images and surface locations on the surface of the subject from which the first scan images are acquired, respectively; extract content from the first scan related to a region of interest in the subject identified in the first scan images; generate a report of results of the first scan based on the extracted content, where the report comprises a surface location on the surface of the subject corresponding to the region of interest and an indication of the region of interest relative to the surface location; automatically create a customized scan protocol for the second scan based on the report, where the customized scan protocol provides a plurality of imaging steps and a plurality of device settings of the second imaging system corresponding to the plurality of imaging steps, respectively; receive second scan images of the subject acquired during the second scan using the second imaging system according to the plurality of imaging steps and the corresponding plurality of device settings of the customized scan protocol; receive second camera images of the surface of the subject acquired during the second scan using at least one second camera associated with the second imaging system; and monitor progress of the customized scan protocol during the second scan to identify performance of the plurality of imaging steps using the second camera images from the at least one second camera.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of system for associating workflows of multiple scans of a subject using different imaging systems, according to a representative embodiment.

FIG. 2 is a flow diagram showing a method of associating workflows of multiple scans of a subject using different imaging systems, according to a representative embodiment.

FIG. 3 is a schematic diagram of a first imaging system and at least one first camera between which correspondence is generated, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, the various embodiments described herein provide a system and method for increasing efficiency and accuracy of diagnostic workflows of follow-up imaging using a second imaging system (e.g., ultrasound) following initial imaging using a first imaging system (e.g., CT scan) by focusing on the areas relevant of a subject to findings from the first imaging system scan. That is, the user (e.g., radiologist, technician, sonographer or other clinician) performing the second scan using the second imaging system is automatically provided findings from the first scan using the first imaging system, and the findings are presented in a manner that associates areas of the subject from the first scan to with the same areas in the second scan, e.g., using cameras. Also, a specific, customized scan protocol for performing the second scan is developed and provided to the technician to focus the second scan on the most relevant regions, as opposed to a generic, standard protocol for the second scan.

For example, cameras may be used for taking pictures of imaging positions and settings of both the first and second imaging systems (e.g., CT and ultrasound) to generate visual surface context for internal anatomical findings. This information is used to automatically populate an imaging results report of a first scan performed by the first imaging system, and to guide creation of a customized scan protocol which is optimized for performing a second scan by the second imaging system.

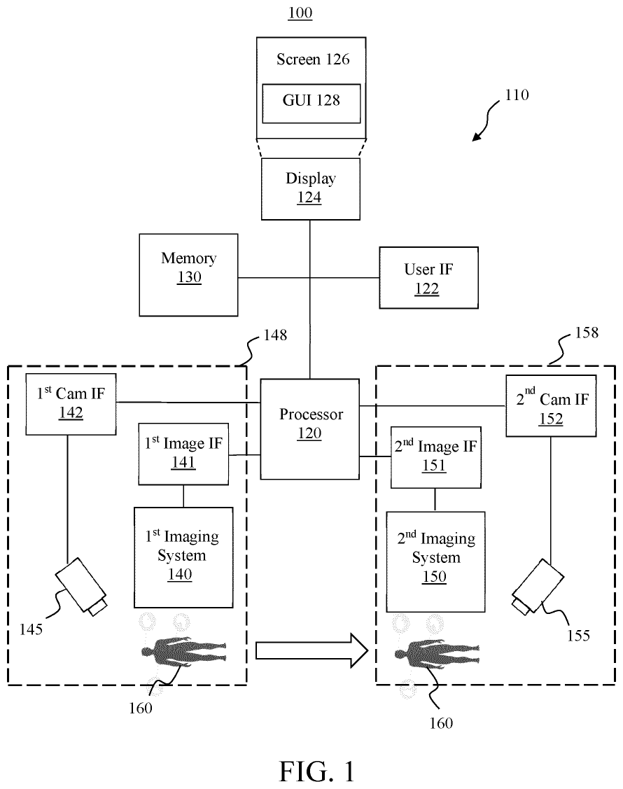

FIG. 1 is a simplified block diagram of system for associating workflows of multiple scans of a subject using different imaging systems, according to a representative embodiment.

Referring to FIG. 1, system 100 includes a processing system 110, and a first imaging system 140 and a second imaging system 150 connected to the processing system 110. The processing system 110 includes a processor 120, a user interface (IF) 122, a display 124, and memory 130. For purposes of illustration, the processing system 110 is depicted as being shared by the first and second imaging systems 140 and 150. However, it is understood that the processing system 110 and the various components therein may be implemented as multiple processing systems, e.g., one of which interfaces with the first imaging system 140 and the other one of which interfaces with the second imaging system 150, where the multiple processing systems may or may not share one or more components, without departing from the scope of the present teachings. Each of first imaging system 140 and the second imaging system 150 may be a CT system, an MRI system, a PET system or an ultrasound imaging system, for example. For purposes of description, it is assumed that the first imaging system 140 is a CT system and the first scan of the subject (e.g., patient) 160 is a CT scan, and that the second imaging system 150 is an ultrasound imaging system and the second scan of the subject 160 is an ultrasound scan.

The processor 120 is representative of one or more processing devices, and may be implemented by a general purpose computer, a central processing unit, a computer processor, a microprocessor, a microcontroller, a state machine, field programmable gate arrays (FPGAs), application specific integrated circuits (ASICs), programmable logic device, or combinations thereof, using any combination of hardware, software, firmware, hard-wired logic circuits, or combinations thereof. The processor 120 may include multiple processors, parallel processors, or both. Multiple processors may be included in, or coupled to, a single device or multiple devices. The term “processor” as used herein encompasses an electronic component able to execute a program or machine executable instruction, and may also refer to a collection of processors within a single computer system or distributed among multiple computer systems, such as in a cloud-based or other multi-site application. Programs have software instructions performed by one or multiple processors that may be within the same computing device or which may be distributed across multiple computing devices.

The memory 130 may include main memory and/or static memory, where such memories may communicate with each other and the processor 120 via one or more buses. The memory 130 may be implemented by any number, type and combination of random access memory (RAM) and read-only memory (ROM), for example, and may store various types of information, such as software algorithms, artificial intelligence (AI) machine learning models, and computer programs, all of which are executable by the processor 120. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, Blu-ray disk, a universal serial bus (USB) drive, or any other form of storage medium known in the art. The memory 130 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 130 may store software instructions and/or computer readable code that enable performance of various functions. The memory 130 may be secure and/or encrypted, or unsecure and/or unencrypted.

The processor 120 and the memory 130 may include or have access to an AI engine or module, which may be implemented as software that provides artificial intelligence, such as natural language processing (NLP) algorithms, and machine learning algorithms, such as neural network modeling, described herein. The AI engine may reside in any of various components in addition to or other than the processor 120, such as the memory 130, an external server, and/or the cloud, for example. When the AI engine is implemented in a cloud, such as at a data center, for example, the AI engine may be connected to the processor 120 via the internet using one or more wired and/or wireless connection(s).

The user interface 122 is configured to provide information and data output by the processor 120 and/or the memory 130 to the user and/or to provide information and data input by the user to the processor 120 and/or the memory 130. That is, the user interface 122 enables the user to enter data and to control or manipulate aspects of the processes described herein, and to control or manipulate aspects of the ultrasound imaging. The user interface 122 also enables the processor 120 to indicate the effects of the user's control or manipulation to the user.

All or a portion of the user interface 122 may be implemented by a graphical user interface (GUI), such as GUI 128 on a touch screen 126 of the display 124, for example. The user interface 122 includes push buttons operable (pushed) by the user to initiate various commands for manipulating the displayed image, making measurements and calculations, and the like during an imaging session (e.g., CT or ultrasound examination). The push buttons may be displayed by the GUI 128 on the touch screen 126, or may be physical buttons, for example. The user interface 122 may further include any other compatible interface devices for performing ultrasound examinations, such as a mouse, a keyboard, a trackball, a joystick, microphone, a video camera, a touchpad, or voice or gesture recognition captured by a microphone or video camera, for example.

The display 124 may be any compatible monitor for displaying ultrasound images, such as a computer monitor, a liquid crystal display (LCD), an organic light emitting diode (OLED), a flat panel display, or a solid-state display, for example, for viewing internal images of the subject 160. The display 124 includes the touch screen 126 and the GUI 128 to enable the user to interact with the displayed images and features.

The first imaging system 140 is located in a first suite 148 for performing diagnostic imaging of the subject 160 during a first scan (exam) in accordance with a first workflow. Also located in the first suite 148 is a first camera 145 associated with the first imaging system 140 and configured to acquire first camera images (e.g., photos and/or videos) of the subject 160 during the first scan by the first imaging system 140. It is understood that the first camera 145 is representative of one or more cameras in the first suite 148. When there is more than one first camera 145, they are arranged in different positions within the first suite 148 and configured to acquire first camera images of different portions of the subject 160 and/or of the same portion of the subject 160 from different angles. The first camera 145 may be a digital still camera, such as compact, bridge, digital single-lens reflex (DSLR), and mirrorless cameras, a digital video camera, a smartphone or tablet cameras, for example. In an example, the first imaging system 140 may be a CT scanning device including x-ray source(s) and x-ray detector(s) positioned within a gantry, in which the subject 160 is positioned. In this case, the first scan is a CT scan, and the first camera 145 is positioned in the gantry to acquire the first camera images of the subject 160 as the subject 160 moves through the gantry. The first camera images acquired by the first camera 145 may be spatially and temporality registered with the first scan images acquired by the first imaging system 140 during the first scan, as discussed below. The user is then able to visually identify the physical position of the subject 160 using the first camera image at the time each of the first scan images is acquired by the first imaging system 140.

The second imaging system 150 is located in a second suite 158 for performing diagnostic imaging of the subject 160 during a second scan (exam) in accordance with a second workflow. Also located in the second suite 158 is a second camera 155 associated with the second imaging system 150 and configured to acquire second camera images (e.g., photos and/or videos) of the subject 160 during the second scan by the second imaging system 150. As discussed above with regard to the first camera 145, it is understood that the second camera 155 is representative of one or more cameras in the second suite 158. When there are more than one second camera 155, they are arranged in different positions within the second suite 158 and configured to acquire second camera images of different portions of the subject 160 and/or of the same portion of the subject 160 from different angles.

In an example, the second imaging system 150 may be an ultrasound imaging device including a transducer probe for transmitting ultrasound waves into the subject 160 and receiving echo information in response, as discussed above. When the second imaging system 150 is an ultrasound imaging system, it further includes a transducer probe having a transducer array with a two-dimensional array of transducers (not shown). The array of transducers is capable of scanning in two or three dimensions for transmitting ultrasound waves into subject 160 within the second suite 158, and receiving echo information in response. The transducer array may include capacitive micromachined ultrasonic transducers (CMUTs) or piezoelectric transducers formed of materials such as PZT or PVDF, for example. The transducer array is coupled to a microbeamformer in the transducer probe, which controls reception of signals by the transducers.

Further, the memory 130 would include a probe interface module (not shown) for interfacing the transducer probe with the processor 120 to control acquisition of ultrasound images of the subject 160. The probe interface module may include a transmit/receive (T/R) switch coupled to the microbeamformer of the transducer probe by a probe cable. The T/R switch switches between transmission and reception modes, e.g., under control of the processor 120 and/or the user interface 122. The processor 120 also controls the directions in which beams are steered and focused via the probe interface module. Beams may be steered straight ahead from (orthogonal to) the transducer array, or at different angles for a wider field of view. The processor 120 may also include a main beamformer that provides final beamforming following digitization. Generally, the transmitting of ultrasound waves and the receiving of echo information is well known, and therefore additional detail in this regard is not included herein.

The transducer probe may be physically manipulated by the user during ultrasound examination. In this case, the second camera 155 is positioned to acquire the second camera images of positions (locations and orientations) of the transducer probe when the transducer probe acquires each of the second scan images during the ultrasound exam. In another example, the second imaging system 150 may be an MRI device including a main magnet formed by superconducting coils, gradient coils, radio frequency (RF) coils, where the subject 160 is positioned within a bore formed by the main magnet. In this case, the second scan is an MRI scan, and the second camera 155 is positioned near the entry of the bore to acquire the second camera images of the subject 160 as the subject 160 moves into the bore.

The second camera images acquired by the second camera 155 are registered with the second scan images acquired by the second imaging system 150 during the second scan. The registration may be based on respective time stamps of the second camera 155 and the second imaging system 150. If the second camera 155 and the second imaging system 150 are separate systems, then time synchronization is performed first. The user is then able to visually identify the physical position of the subject 160 and/or the position of the second imaging system 150 using the second camera images at the time each of the second scan images is acquired by the second imaging system 150. For example, when the second imaging system 150 is an ultrasound imaging system, a second camera image acquired by the second camera 155 of each probe position may be linked to an ultrasound image(s) acquired by the probe at that probe position.

The system 100 further includes a first image IF 141 interfacing the first imaging system 140 with the processor 120, and a first camera IF 142 interfacing the first camera 145 with the processor 120. For example, the first imaging system 140 may send image data of the first scan images to the processor 120 and receive control commends from the processor 120 (e.g., adjusting imaging parameters, triggering image acquisitions) via the first image IF 141, and the first camera 145 may send image data of the first camera images to the processor 120 and receive control commends from the processor 120 (e.g., adjusting parameters, triggering camera image acquisitions) via the first camera IF 142. The first image IF 141 and the first camera IF 142 may be located in the first suite 148 (as shown) or in the processing system 110.

Likewise, the system 100 includes a second image IF 151 interfacing the second imaging system 150 with the processor 120, and a second camera IF 152 interfacing the second camera 155 with the processor 120. For example, the second imaging system 150 may send image data of the second scan images to the processor 120 and receive control commends from the processor 120 (e.g., adjusting imaging parameters, triggering image acquisitions, receiving customized scan protocol instructions) via the second image IF 151, and the second camera 155 may send image data of the second camera images to the processor 120 and receive control commands from the processor 120 (e.g., adjusting parameters, triggering camera image acquisitions) via the second camera IF 152. The second image IF 151 and the second camera IF 152 may be located in the second suite 158 (as shown) or in the processing system 110. The first and second imaging systems 140, 150 and the first and second cameras 145, 155 may communicate with the processor 120 via the respective interfaces over wired or wireless network connections.

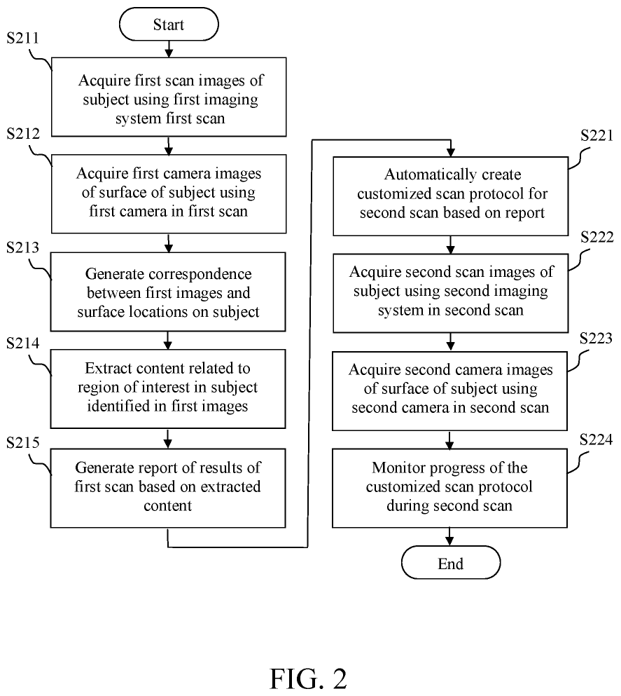

As discussed above, the memory 130 stores instructions executable by the processor 120. When executed, the instructions cause the processor 120 to implement one or more processes for associating workflows of multiple scans of a subject using different imaging systems, described below with reference to FIG. 2, for example, as well as to control performance of the first and second imaging systems 140 and 150.

FIG. 2 is a flow diagram showing a method of associating workflows of multiple scans of a subject using different imaging systems, according to a representative embodiment. The method may be implemented by the system 100, discussed above, under control of the processor 120 executing instructions stored as the various software modules in the memory 130, for example. Blocks S211 to S215 of FIG. 2 are performed using a first imaging system (e.g., first imaging system 140) and at least one first camera (e.g., first camera 145) associated with the first imaging system, as well as image data respectively acquired by the same. Blocks S221 to S224 are performed using a second imaging system (e.g., second imaging system 150) and at least one second camera (e.g., second camera 155) associated with the second imaging system, as well as image data respectively acquired by the same.

Referring to FIG. 2, first scan images (e.g., diagnostic images) of the subject are acquired during a first scan using the first imaging system in block S211. The first scan is performed according to a first workflow, which may be established for the type of first imaging system according to industry and/or institutional standards, for example, as would be apparent to one skilled in the art.

In block S212, first camera images of a surface of the subject are acquired during the first scan using the at least one first camera associated with the first imaging system. In an embodiment, the at least one first camera may be triggered to acquire the first camera images by actuation of the first imaging system to acquire the first scan images. In this case, one first camera image is associated with each of the first scan images during the first scan.

In block S213, a correspondence is generated between the first scan images acquired by the first imaging system and surface locations on the surface of the subject from which the first scan images are acquired, respectively. In an embodiment, generating the correspondence between the first scan images and the respective surface locations on the surface of the subject may include performing spatial registration between a location of the at least one first camera and a scan plane of the first imaging system, and performing temporal registration between camera frames of the first camera images of the surface of the subject acquired by the at least one first camera during the first scan and the first scan images of the subject acquired by the first imaging system during the first scan.

For example, for CT and MRI scans as the first imaging system, the scan plane may be one of a transverse, sagittal or coronal plane relative to the subject. The spatial locations of the at least one camera and the scan plane of the first imaging system may be previously identified in a common three-dimensional space provided for the first suite (e.g., first suite 148) in which they are positioned. Alternatively, the at least one camera and/or the first imaging system may include position tracking devices, such as electromagnetic (EM) sensors, inertial measurement unit (IMU) sensors, or optical shape sensing devices, for detecting physical positions relative to one another. The spatial registration between the location of the at least one first camera and the scan plane of the first imaging system may be performed once prior to the first scan. For example, the spatial registration may be performed once, e.g., during an initial calibration stage, following installation of the at least one first camera and the first imaging system in the first suite, since the positions will not change during subsequent scans performed by the first imaging system.

The temporal registration may be performed inherently by running the at least one first camera and the first imaging system off of a common system clock. Alternatively, the temporal registration may be performed explicitly by registering the time base of the at least one first camera and the time base of the first imaging system. The temporal registration is performed for each of the first scans by the first imaging system.

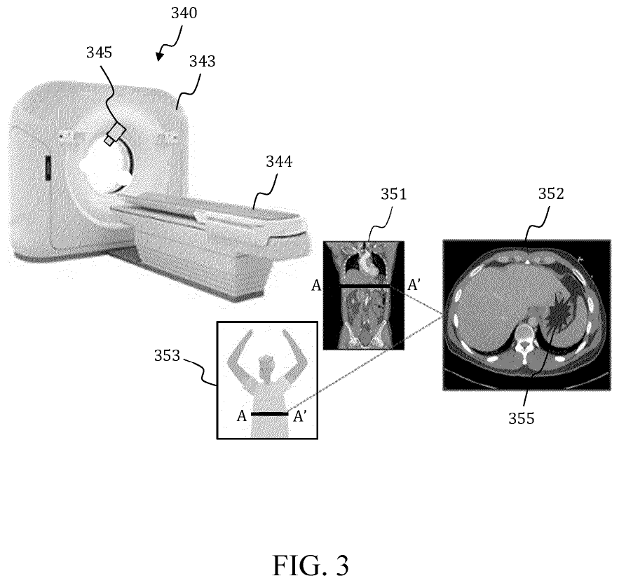

FIG. 3 is a schematic diagram of a first imaging system and at least one first camera between which correspondence is generated, according to a representative embodiment.

Referring to FIG. 3, the first imaging system is a CT scanner 340 for purposes of illustration. The at least one first camera is represented by first camera 345 positioned on the interior of the gantry 343 of the CT scanner 340 for acquiring first camera images of the subject during the first scan when the subject is positioned on table 344 within the gantry. The first camera 345 may be positioned elsewhere in the first suite, such as the ceiling or a wall, without departing from the scope of the present teachings.

The CT scanner 340 acquires illustrative first scan images 351 and 352, which are the coronal and transverse views, respectively. In particular, first scan image 352 is a cross-sectional view of the first scan image 351 along a plane indicated by line A-A′. The first camera 345 acquires a first camera image 353 of the subject for a viewpoint similar to that of the first scan image 351. In response to the spatial registration between the location of the first camera 345 and a scan plane of the CT scanner 340 (where the scan plane is typically transverse to a plane of the table 334), the first camera image 353 is spatially aligned with the first scan image 351 such that the line A-A′ matches in both images. In response to the temporal registration, the time when first camera image 353 was acquired by the first camera 345 matches the time when the first scan image 352 was acquired by the CT scanner 340.

Referring again to FIG. 2, in block S214, content is extracted from the first scan related to a region of interest in the subject identified in the first scan images. The region of interest may be a tumor, cyst, growth, foreign object, organ, medical instrument, or other internal physical structure visible in the first scan images. For example, FIG. 3 shows an illustrative region of interest 355 (e.g., tumor) in the first scan image 352.

The content may be extracted from the first scan by detecting and localizing the region of interest in one or more of the first scan images. For example, the content may be extracted using automated shape recognition techniques in the one or more first scan images. The shape recognition techniques may include edge detection, brightness and color detection, and texture recognition, for example, as are well known to one skilled in the art.

Alternatively, or in addition, the content may be extracted by applying a first machine learning algorithm, such as convolutional neural network (CNN), a recurrent neural network (RNN), an artificial neural network (ANN), or a transformer network, for example. The first machine learning algorithm may be initially trained using previous images of subjects acquired by the first imaging system (or other imaging systems of the same type as the first imaging system from other medical facilities) during corresponding previous scans. The training images may be retrieved from one or more image databases (not shown), such as a picture archiving and communication system (PACS) database and/or a radiology information system (RIS) database, for example. During the training, image features are associated with various types of regions of interest. For example, the training dataset of training images may include a large variety of previous images of clinical features of interest. In one approach, an object detection model may be trained to draw bounding boxes around the features of interest. The ground truth locations and bounds of these features of interest in the training images may be provided by clinical experts. In another approach, a classification network model may be trained to differentiate between different distinct classes of features of interest. For each class, sufficient training images (including a “negative” or “reference” class in which no relevant/suspicious features of interest are present) are used to train the first machine learning algorithm in a balanced manner. The training enables the first machine learning algorithm to identify relevant regions of interest in current first scan images.

Optionally, the content may be identified and extracted manually by the user reviewing the first scan. For example, one or more first scan images may be displayed on a display (e.g., display 124) for viewing by the user, and the user may identify the region of interest on the display using a GUI (e.g., GUI 128).

In block S215, a report of results of the first scan is generated based on the extracted content. Generating the results report may include associating the first camera images with the first scan images, respectively, estimating a surface location on the surface of the subject corresponding to the region of interest using the first scan images and the first camera images associated with the first scan images, and indicating in the results report the region of interest in the subject relative to the estimated surface location. Associating the first camera images with the first scan images may be performed using the spatial and temporal registration of the first camera images with the first scan images discussed above with reference to block S213. The surface location on the surface of the subject corresponding to the region of interest may be estimated based on the correspondence between the first scan images (e.g., first scan image 352) and the locations of the imaged slice in the first camera images (e.g., first camera image 353 at line A-A′), for example. The results report may be stored in a memory (e.g., memory 130) common to or accessible by both of the first and second imaging systems, as well as the processor(s) described herein.

The extracted content from block S214 may be tagged in the results report with corresponding surface locations, as well as other locating information, such as anterior-posterior (A-P) coordinates (e.g., as in first scan image 352) and/or depth from skin surface, for example. For example, the results report may include text such as: “tumor present in right liver lobe, 9 cm from anterior surface, 4 cm from lateral surface.” The results report may be generated in a format that is readable by protocoling software, discussed below.

In block S221, a customized scan protocol for the second imaging system to perform the second scan is automatically created based on the results report. That is, the customized scan protocol provides imaging steps and corresponding device settings of the second imaging system, respectively, to perform the second scan according to an optimized second workflow.

The customized scan protocol may be created by customizing a predetermined workflow based on the first workflow, as discussed below. For example, the second imaging system may store previously created workflows for imaging different organs and regions of the body, which may be provided by clinical experts. The customized scan protocol may be obtained by identifying the applicable predetermined workflow and modifying it based on information in the results report. This may include reordering and/or eliminating steps of the applicable predetermined workflow so that steps directed to the region of interest and/or the corresponding surface location determined in blocks S214 and S215 are prioritized. The customized scan protocol thus improves efficiency, e.g., by eliminating or subjugating imaging steps involving other organs or regions of the body not highlighted in the result report. When the second imaging system is an ultrasound system, for example, the customized scan protocol provides step-by-step instructions for the user to acquire images using the transducer probe positioned at relevant locations on the surface of the subject for capturing the region of interest identified by the first scan, where the instructions also include probe settings (e.g., gain, penetration depth, focal zone, bandwidth, aperture) to be applied at each of the locations for each image.

Creating the customized scan protocol for the second scan may include reading the results report at the second imaging system and/or the processor(s), and creating the customized scan protocol using protocoling software, e.g., stored in the memory 130. The protocoling software may step the user through different acquisitions, measurements, annotation labels and the like for generating the second scan, e.g., per the requirements of the medical facility. Notably, the results report may be populated in a format understandable by the protocoling software, so that the customized scan protocol may be auto-generated in the appropriate order. The format of the results report may be customizable based on specific needs of the protocoling software on the second imaging system platform. In an embodiment, the format may be chosen by the user based on which options exist for the second imaging system at the particular institution, and may default to a predetermined format as well.

The customized scan protocol may be created to first cover the relevant anatomical regions mentioned in the results report in a sequential manner, followed by surrounding regions not mentioned in the results report, for completeness. For example, when the results report indicates the presence of a tumor in the left liver lobe 5 cm from the surface of the subject, and another tumor in the right liver lobe 9 cm from the surface of the subject, the customized scan protocol is created for the second imaging system to first scan the left liver lobe, followed by the right liver lobe, and then eventually other regions of the abdomen not specifically mentioned in the results report (to rule out other findings). The customized scan protocol may also be optimized based on a body surface map of the subject obtained from the first imaging system (e.g., CT system) during the first scan. For example, when the subject has a higher than average BMI, then the customized scan protocol may be optimized to compensate accordingly by adjusting acquisition parameters (e.g., gain) of the second imaging system (e.g., ultrasound imaging system).

In an embodiment, reading the results report using the protocoling software may include performing natural language processing (NLP) on text of the report. The NLP can either be part of the protocoling software or it can be a separate algorithm that processes the results report and feeds the NLP output into the protocoling software. The NLP may be performed by an NLP algorithm using word embedding technology, for example, to identify descriptive text describing first scan images showing the region of interest, and to convert the descriptive text to computer readable data. The protocoling software uses terms in the descriptive text to provide the step-by-step instructions for the second scan. NLP is well known, and may include syntax and semantic analyses, for example, and deep learning for improving understanding by the NLP algorithm with the accumulation of data, as would be apparent to one skilled in the art. In various embodiments, all or part of the processes provided by the NLP algorithm may be implemented by an AI engine, for example, executed by the processor 120, described above.

In another embodiment, reading the results report using the protocoling software may include populating a look-up table using tags applied while generating the results report in block S215. For example, the tags may be output by the NLP algorithm, for example. The tags may provide a link between the feature and its approximate surface location on the subject. This provides an easy interface for the protocoling software to suggest the appropriate scanning steps. The look-up table associates the tags with predetermined descriptive terms, which are used by the protocoling software to provide the step-by-step instructions for the second scan. For example, if the tags indicate that there is one feature in the left lower abdominal quadrant and one feature in the left upper abdominal quadrant, then the protocoling software will prioritize scanning the left side of the subject based on the tags before progressing to the right side for completeness.

In block S222, second scan images of the subject are acquired during the second scan using the second imaging system. The second scan is performed according to the second workflow, which includes the imaging steps and the corresponding device settings of the customized scan protocol created in block S221. In an embodiment, actual device settings of the second imaging system may be automatically prepopulated for use in the second scan according to the imaging steps and corresponding device settings of the customized scan protocol. The actual device settings of the second imaging system are then automatically adjusted for each of the imaging steps during the second scan, in accordance with the prepopulated actual device settings.

In block S223, second camera images of the surface of the subject are acquired during the second scan using at least one second camera (e.g., second camera 155). The at least one second camera is associated with the second imaging system. In an embodiment, the at least one second camera may triggered to acquire the second camera images by actuation of the second imaging system to acquire the second scan images. In this case, one second camera image is associated with each of the second scan images during the second scan.

In block S224, progress of the customized scan protocol is monitored during the second scan using the second camera images from the at least one second camera. The progress is monitored in order to identify performance of the imaging steps in the second workflow. In an embodiment, as each step of the customized scan protocol is completed, the device settings of the second imaging system are automatically adjusted for the next step in the customized scan protocol, thus aid in execution of the second workflow. The user may override the customized scan protocol or the device settings at any point in the process to proceed manually with the second scan.

As an example, the progress of the customized scan protocol may be monitored using a second machine learning algorithm, such as a CNN, an RNN, an ANN, or a transformer network, for example. The second machine learning algorithm may be initially trained using previous images of subjects acquired by the second imaging system (or other imaging systems of the same type as the second imaging system from other medical facilities) from different locations on subject during corresponding previous scans, and previous camera images acquired by the at least one second camera (or other cameras imaging procedures of the same type as the second imaging system from other medical facilities), together with corresponding body poses of the subject, as would be apparent to one skilled in the art. For example, when the second imaging system is an ultrasound imaging system, the second machine learning algorithm may be trained with second camera images of the subject with the probe in the field of view (FOV), with ground truth labels indicating the probe position relative to the subject (e.g., which quadrant the probe is in). During clinical operation, when the probe is detected in a particular location and second scan images have been saved by the user, then the user may be prompted to move to the next step in the customized scan protocol. When the user moves the probe to the next location suggested in the next step of the customized scan protocol, then the movement can be detected from the second camera images. Accordingly, the second image settings for the next step in the customized scan protocol are updated automatically. The training images may be retrieved from one or more image databases (not shown), such as a PACS database and/or a RIS database, for example. The training enables the second machine learning algorithm to identify imaging steps in current second scan images based on the positioning of the second imaging system and the subject.

In clinical operation, the second machine learning algorithm may estimate where in the second workflow of the second imaging system the user currently is. For example, when the customized scan protocol includes one portion directed to scanning the left side of the subject's abdomen and another portion directed to scanning the right side of the subject's abdomen, the second machine learning algorithm linked to the at least one second camera will automatically indicate when the user transitions from the left side of the subject to the right side, i.e., from one portion of the customized scan protocol to the other portion of the customized scan protocol. This automatic detection may trigger the choice of the device settings created in block S221 for the second system to support proper execution of the customized scan protocol.

Optionally, to improve performance, the monitoring of the customized scan protocol during the second scan using the at least one second camera may be augmented by a classification network that operates on the second scan images that have been acquired. The classification network differentiates between different distinct classes of features of interest in the second scan images. In addition, the results from the first imaging system may be used as a comparator to confirm that the results of the second scan are capturing the relevant features as reported by the first scan. Completeness of each step of the customized scan protocol may be estimated by analyzing the second camera images together with the second scan images acquired by the second imaging system.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Although associating workflows of multiple scans of a subject using different imaging systems has been described with reference to exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Also, although associating workflows of multiple scans of a subject using different imaging systems has been described with reference to particular means, materials and embodiments, there is no intention to be limited to the particulars disclosed; rather the embodiments extend to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of performing a first scan of a subject using a first imaging system and a second scan of the subject using a second imaging system, the method comprising:
acquiring first scan images of the subject during the first scan using the first imaging system;
acquiring first camera images of a surface of the subject during the first scan using at least one first camera associated with the first imaging system;
generating a correspondence between the first scan images acquired by the first imaging system and surface locations on the surface of the subject from which the first scan images are acquired, respectively;
extracting content from the first scan related to a region of interest in the subject identified in the first scan images;
generating a report of results of the first scan based on the extracted content, wherein the report comprises a surface location on the surface of the subject corresponding to the region of interest and an indication of the region of interest relative to the surface location;
automatically creating a customized scan protocol for the second scan based on the report, wherein the customized scan protocol provides a plurality of imaging steps and a plurality of device settings of the second imaging system corresponding to the plurality of imaging steps, respectively;
acquiring second scan images of the subject during the second scan using the second imaging system according to the plurality of imaging steps and the corresponding plurality of device settings of the customized scan protocol;
acquiring second camera images of the surface of the subject during the second scan using at least one second camera associated with the second imaging system; and
monitoring progress of the customized scan protocol during the second scan to identify performance of the plurality of imaging steps using the second camera images from the at least one second camera.

2. The method of claim 1, wherein generating the report of results comprises:
associating the first camera images with the first scan images, respectively;
estimating the surface location on the surface of the subject corresponding to the region of interest using the first scan images and the first camera images associated with the first scan images; and
determining the region of interest in the subject relative to the estimated surface location.

3. The method of claim 1, wherein generating the correspondence between the first scan images and the respective locations in the subject from which the first scan images are obtained comprises:
performing spatial registration between a location of the at least one first camera and a scan plane of the first imaging system; and
performing temporal registration between camera frames of the first camera images of the surface of the subject acquired by the at least one first camera during the first scan and the first scan images of the subject acquired by the first imaging system during the first scan,
wherein the temporal registration is performed inherently by running the at least one first camera and the first imaging system off of the same system clock, or is performed explicitly by registering respective time bases of the at least one first camera and the first imaging system.

4. The method of claim 3, wherein the spatial registration between the location of the at least one first camera and the scan plane of the first imaging system is performed once following installation of the at least one first camera in a suite of the first imaging system.

5. The method of claim 1, extracting the content from the first scan comprises detecting and localizing the region of interest in one or more of the first scan images using automated shape recognition techniques and/or a machine learning algorithm applying an artificial neural network (ANN), a convolutional neural network (CNN), or a recurrent neural network (RNN).

6. The method of claim 1, wherein creating the customized scan protocol for the second scan based on the report comprises:
reading the report using protocoling software; and
creating the customized scan protocol using the protocoling software.

7. The method of claim 6, wherein reading the report using the protocoling software comprises performing natural language processing (NPL) on text of the report.

8. The method of claim 6, wherein reading the report using the protocoling software comprises populating a look-up table using tags applied while generating the report, wherein the look-up table associates the tags with predetermined descriptive terms.

9. The method of claim 1, further comprising:
prepopulating actual device settings of the second imaging system for use in the second scan according to the plurality of device settings of the second imaging system corresponding to the plurality of imaging steps; and
automatically adjusting the actual device settings of the second imaging system for the plurality of imaging steps during the second scan according to the prepopulated actual device settings.

10. A system comprising:
a first imaging system configured to acquire first scan images of a subject during a first scan;
a second imaging system configured to acquire second scan images of the subject during a second scan subsequent to the first scan;
at least one first camera associated with the first imaging system and configured to acquire first camera images of a surface of the subject during the first scan;
at least one second camera associated with the second imaging system and configured to acquire second camera images of the surface of the subject during the second scan;
at least one computer processor; and
at least one memory storing instructions for associating workflows of the first scan and the second scan, wherein when executed, the instructions cause the at least one computer processor to:
generate a correspondence between the first scan images acquired by the first imaging system and surface locations on the surface of the subject from which the first scan images are acquired during the first scan, respectively;
extract content from the first scan related to a region of interest in the subject identified in the first scan images;
generate a report of results of the first scan based on the extracted content, wherein the report is generated by associating the first camera images with the first scan images, respectively, estimating a surface location on the surface of the subject corresponding to the region of interest using the first scan images and the first camera images associated with the first scan images, and indicating in the report the region of interest in the subject relative to the estimated surface location;
create a customized scan protocol for the second scan based on the report, wherein the customized scan protocol provides a plurality of imaging steps and a plurality of device settings of the second imaging system corresponding to the plurality of imaging steps, respectively, wherein the second scan images of the subject are acquired during the second scan by the second imaging system according to the plurality of imaging steps and the corresponding plurality of device settings of the customized scan protocol; and
monitor progress of the customized scan protocol during the second scan to identify performance of the plurality of imaging steps using the second camera images from the at least one second camera.

11. The system of claim 10, wherein generating the correspondence between the first scan images and the respective locations in the subject from which the first scan images are obtained comprises:
performing spatial registration between a location of the at least one first camera and a scan plane of the first imaging system; and
performing temporal registration between camera frames of the first camera images of the surface of the subject acquired by the at least one first camera during the first scan and the first scan images of the subject acquired by the first imaging system during the first scan.

12. The system of claim 10, wherein creating the customized scan protocol for the second scan based on the report comprises:
reading the report using protocoling software; and
creating the customized scan protocol using the protocoling software.

13. The system of claim 12, wherein reading the report using the protocoling software comprises performing natural language processing (NPL) on text of the report.

14. The system of claim 12, wherein reading the report using the protocoling software comprises populating a look-up table using tags applied while generating the report, wherein the look-up table associates the tags with predetermined descriptive terms.

15. The system of claim 10, wherein the instructions further cause the at least one processor to:
prepopulate actual device settings of the second imaging system for use in the second scan according to the plurality of device settings of the second imaging system corresponding to the plurality of imaging steps; and
automatically adjust the actual device settings of the second imaging system for the plurality of imaging steps during the second scan according to the prepopulated actual device settings.

16. The system of claim 10, wherein the first imaging system comprises computed tomography (CT) imaging and the second imaging system comprises ultrasound imaging.

17. The system of claim 10, wherein the first imaging system comprises magnetic resonance imaging (MRI) and the second imaging system comprises ultrasound imaging.

18. A non-transitory computer readable medium storing instructions for performing a first scan of a subject using a first imaging system and a second scan of the subject using a second imaging system that, when executed by at least one processor, cause the at least one processor to:
receive first scan images of the subject acquired during the first scan using the first imaging system;
receive first camera images of a surface of the subject acquired during the first scan using at least one first camera associated with the first imaging system;
generate a correspondence between the first scan images and surface locations on the surface of the subject from which the first scan images are acquired, respectively;
extract content from the first scan related to a region of interest in the subject identified in the first scan images;
generate a report of results of the first scan based on the extracted content, wherein the report comprises a surface location on the surface of the subject corresponding to the region of interest and an indication of the region of interest relative to the surface location;
automatically create a customized scan protocol for the second scan based on the report, wherein the customized scan protocol provides a plurality of imaging steps and a plurality of device settings of the second imaging system corresponding to the plurality of imaging steps, respectively;
receive second scan images of the subject acquired during the second scan using the second imaging system according to the plurality of imaging steps and the corresponding plurality of device settings of the customized scan protocol;
receive second camera images of the surface of the subject acquired during the second scan using at least one second camera associated with the second imaging system; and
monitor progress of the customized scan protocol during the second scan to identify performance of the plurality of imaging steps using the second camera images from the at least one second camera.

19. The non-transitory computer readable medium of claim 18, wherein the instructions cause the at least one processor to generate the report of results by:
associating the first camera images with the first scan images, respectively;
estimating the surface location on the surface of the subject corresponding to the region of interest using the first scan images and the first camera images associated with the first scan images; and
determining the region of interest in the subject relative to the estimated surface location.

20. The non-transitory computer readable medium of claim 18, wherein the instructions cause the at least one processor to generate the correspondence between the first scan images and the respective locations in the subject from which the first scan images are obtained by:
performing spatial registration between a location of the at least one first camera and a scan plane of the first imaging system; and
performing temporal registration between camera frames of the first camera images of the surface of the subject acquired by the at least one first camera during the first scan and the first scan images of the subject acquired by the first imaging system during the first scan.
